(19) United States
(12) Patent Application Publication
(10) Pub. No.: US 2011/0237888 A1
(43) Pub. Date: Sep. 29, 2011

(54) GUIDE TUBE FOR GUIDING ENDOSCOPE OR SURGICAL TOOL IN OR INTO BODY CAVITY

(76) Inventor: Motohiko MATSUSHITA, Kanagawa (JP)

(21) Appl. No.: 13/015,989

(22) Filed: Jan. 28, 2011

(30) Foreign Application Priority Data
Mar. 23, 2010 (JP) 2010-066241

(51) Int. Cl. A61B 1/00

(52) U.S. Cl. 600/114

(57) ABSTRACT

In order to secure a path of insertion for an insertion rod 2 of an endoscope 1 and for transformation from a flexible structure to a rigid structure, a guide tube is constituted by a flexible double tube 13 having flexible inner and outer tubes 11 and 12 which are flexible in bending directions and fitted one on the other to define a hermetically closed space therebetween. Muscle members 14, each composed of a friction strip 15 and attached with a metal wire 16, are provided at predetermined angular positions between the inner and outer tubes. Dislodgements of the muscle members 14 from the respective angular positions are prevented by restraint rings 17. A suction tube 18 of a pump unit 19 is connected to the double tube 13 to evacuate a fluid from the closed space between the inner and outer tubes 11 and 12.

### FIELD OF THE INVENTION

This invention relates to a guide tube suitable for use in endoscopy for stably guiding an endoscope or a surgical tool or the like in or into a body cavity of a patient or examinee.

### BACKGROUND OF THE INVENTION

Normally, an endoscope is introduced into a patient along a path of insertion like an upper or lower digestive tract. As shown in Japanese Laid-Open Patent Application 2008-502421, for example, there is a technique of stomach perforation for bypassing an insertion rod of an endoscope from the stomach to the transverse colon of the large intestine. In such a case, it is important to suppress the invasive attacks to a minimum. Therefore, in some cases, it is found extremely difficult to open a path of insertion in such a way as to ensure smooth introduction of an insertion rod of an endoscope for a treatment in an endoscopic operation.

Particularly, in case the insertion rod of the endoscope has a flexible structure, difficulties are often encountered in passing the insertion rod along a path of insertion opened up by perforation and in keeping the insertion rod in a stabilized state after insertion into a target intracavitary site to conduct a precision treatment in an appropriate manner. Further, in lower endoscopy, in the course of introducing an endoscopic insertion rod into a patient along a lower intestinal path, it may become necessary to adjust a path of insertion, for example, by forcibly straightening up the sigmoid colon to let the endoscopic insertion rod pass therethrough.

In this regard, proposed in Japanese Laid-Open Patent Application 2008-502421 is an overcoat tube to be used as a guide tube in establishing a path of insertion for an endoscopic insertion rod. An overcoat tube of this sort is required to have suitable flexibility until it establishes a path of insertion. Of course, it needs to have a greater degree of stiffness than an endoscopic insertion rod in performing the function of securing a path of insertion for the endoscope. After securing a path of insertion as far as a target intracavitary site, the overcoat tube should desirably have increased stiffness to facilitate introduction of the endoscope and to retain a surgical or biopsy tool in a stabilized state during an endoscopic operation or inspection.

That is to say, a guide tube to be used for securing a path of insertion in endoscopy is desired to be variable in stiffness depending upon situations. In Japanese Laid-Open Patent Application 2008-502421 mentioned above, the overcoat tube is not arranged to be variable in stiffness. For example, a guide tube of variable stiffness is disclosed in Japanese Laid-Open Patent Applications 2005-318956 and H-5-503434.

In Japanese Laid-Open Patent Application 2005-318956, a guide tube of variable stiffness is constituted by a series of successively connected ring members each formed with an inclined surface at opposite ends, and a plural number of manipulation wires threaded through the respective ring members. When the manipulation wires are slackened to leave the inclined end faces of the ring members free of a tightening force, the guide tube has flexibility in bending directions. On the other hand, when a tensile force is applied to the manipulation wires, the inclined end faces of preceding and succeeding ring members are brought into tightly pressed engagement with each other to impart a high rigidity to the variable rigidity tube. In the case of Japanese Laid-Open Patent H5-503434 (PCT), a variable rigidity tube is in the form of a double tube structure including inner and outer tubes which are provided with axial grooves and ribs on outer and inner peripheral surfaces, respectively, and fitted one in the other in such a way as to form a hermetically closed space therebetween. When the hermetically closed spaces are retained at the atmospheric pressure, separating the two tubes away from each other, the guide tube has high flexibility in bending directions. When a negative pressure is applied to the closed space, bringing the grooves and ribs into meshing engagement with each other, a high rigidity is imparted to the guide tube.

The variable rigidity tube of Japanese Laid-Open Patent Application 2005-318956, which is composed of a series of successively connected ring members, has a disadvantage in that it requires a large number of parts. Especially, in order to ensure smooth bending movements of the tube, it becomes necessary for the variable rigidity tube to employ a large number of ring members of a short length and to elongate the length of the variable rigidity tube correspondingly to the length of a path of insertion of an endoscope. That is to say, a variable rigidity tube has to be assembled by the use of a great number of ring members.

On the other hand, in the case of the variable rigidity tube of Japanese Laid-Open Patent Application H5-503434 (PCT), having axial grooves and ribs on the outer and inner peripheries of inner and outer tubes, the axial grooves and ribs sometimes fail to fall correctly into meshing engagement with each other for transformation to a rigid tube structure if the tube is flexed in a curved shape at the time of application of a negative pressure to the hermetically closed spaces. Besides, the provision of axial grooves and ribs of predetermined height and depth on both of the inner and outer tubes invariably results in a variable rigidity tube which is unduly large in thickness and diameter.

### SUMMARY OF THE INVENTION

With the foregoing in view, it is an object of the present invention to provide a guide tube for guiding an endoscope or a surgical tool in or into a body cavity of a patient or examinee, the guide tube being simple in construction and small in outside diameter and yet capable of transformation from a flexible structure to a highly rigid structure in a secure and reliable manner.

In order to achieve the above-stated objective, according to the present invention, there is provided a guide tube for guiding an endoscope or a surgical tool in or into a body cavity, which comprises: an inner tube having open fore and rear ends and a longitudinal axis, along with flexibility in bending directions; an outer tube disposed in such a way as to enshroud the inner tube and directly or indirectly connected to fore and rear ends of the inner tube, the outer tube having flexibility in bending directions; at least one of elongated muscle member located in a hermetically closed space defined between the inner and outer tubes in parallel relation with the longitudinal axis of the inner tube, and having an engaging surface on inner or outer side to be brought into pressed engagement with at least one of the inner and outer tubes, the muscle member being attached to an elongated strength retention member having no elasticity in longitudinal direction but having flexibility in bending directions; a restraint member provided at least on one of the inner and outer tubes and adapted to restrain the muscle member of dislodgement in a circumferential direction about the longitudinal axis from a predetermined angular or radial position on the inner tube; and a fluid evacuation means connected to the guide tube for communication with the hermetically closed space between the inner and outer tubes, the evacuation means being switched on to evacuate a fluid from the closed space, bringing the engaging surface into pressed engagement with the inner or outer tube to transform the guide tube into a highly rigid structure, and switched off at the time of restoring flexibility of said guide tube by introduction of a fluid into the closed space.

The inner tube is of a round cylindrical shape having opened fore and rear ends and a longitudinal axis, and having flexibility in bending directions, and further designed to be fitted on an insertion rod of an endoscope. No matter whether it is in an existing path of insertion or in a newly opened path, even if the path is turned in complicated directions, the guide tube should have an internal passage which permits smooth insertion of an endoscope. For this purpose, the inner tube should have a structure which is satisfactory in shape retention capability and yet suitably flexible in bending directions. In consideration of these points, it is desirable for the inner tube to have an alternately raised and sunken surface on the outer peripheral side and a smooth surface on the inner peripheral side in a sectional view in the longitudinal direction. For example, an alternately raised and sunken surface may be formed by successively connecting ring-like members or by the use of a helically wound wire. Of course, it is possible to adopt other arrangements such as, for example, embedment of a helical metal wire in a flexible synthetic resin tube or use of a bellows structure.

The outer tube should have flexibility in bending directions but does not need to have shape retaining properties. From the standpoint of reduction of the outside diameter, it is desired to be in the form of a thin wall tube. On the inner side, the outer tube may have either a roughened surface or a smooth surface, but is it desired to have a slippery surface on the outer side.

Muscle members which are interposed between the inner and outer tubes are each constituted by a friction strip having an engaging surface or surfaces of a predetermined width, and a strength retention member which is integrally attached on the friction strip. In this instance, the engaging surface is brought into frictional engagement with one or both of the inner and outer tubes thereby to keep the muscle member from positional deviations relative to the inner or outer tube. In case one of the inner and outer tubes is formed of a shape retaining material while the other one is formed of a thin wall material, the muscle member is brought into engagement with the shape retaining material in such a way as to prevent relative positional deviations. The strength retention member can be constituted by a non-elastic or barely elastic linear material (e.g., a metal wire or carbon fiber) which is embedded in a frictional strip. The strength retention member is preferred to be a metal wire of a round shape in cross section which does not have any particular directivity in bending action. In a free state or when not in intimate contact with the inner and outer tubes, the muscle member has a certain degree of flexibility in bending directions. When the muscle member is arrested in a restrained state between the inner and outer tubes, the non-elastic strength retention member becomes a resistance to a bending force and transforms the double tube into a highly rigid structure. Accordingly, the greater the number of the muscle members, the larger becomes the difference between the initial flexible structure and the transformed rigid structure of the guide tube. In addition, the broader becomes the range of directivity. However, since the muscle members are located at intervals in the circumferential direction, provision of an excessively large number of muscle members can result in complication of the guide tube construction. In this regard, it suffices to provide a few muscle members, preferably, three or four muscle members to rigidify the double tube in all directions.

For example, the strength retention member may be constituted by a hollow tubular member like a coil tube of a tightly wound metal wire. In this case, a manipulation wire which is fixed at its fore end is threaded through the tubular member and led out at the rear end of the guide tube. By adoption of this arrangement, it becomes possible to control the degree of flexure of the flexible double tube which is located in a path of insertion or in a body cavity, straightening up the double tube in a curved shape by applying a tensile force to the manipulation wire. For controlling the flexure of the double tube, the manipulation wire is not necessarily required to be provided in association with a muscle member. For instance, a manipulation wire may be provided independently of the strength retention member of the muscle member, fixing the fore end of the manipulation wire to the fore end of the double tube and connecting the rear end of the wire to a manipulation means to push or pull the wire in the axial direction at a predetermined radial or angular position in the circumferential direction of the double tube to straighten up the double tube which is left in a curved shape.

The double tube which is composed of the above-described inner and outer tubes is freely bendable along a path of insertion in a body cavity. As the double tube is flexed in a curved shape at a turn of a sinuous path of insertion, a muscle member is relatively stretched out on the inner side of the flexure having a smaller radius of curvature, while a muscle member is relatively shrunk in on the outer side of the flexure having a larger radius of curvature. Therefore, each muscle member should be allowed to move axially in a forward or rearward direction. Preferably, each muscle member should be formed in a shorter length as compared with the flexible double tube. The length of the muscle member is determined in consideration of curvatures in a path of insertion in which the guide tube is to be placed. Further, the muscle members need to be located constantly at the fore end of the flexible double tube. Accordingly, it is desirable to fix each muscle member to the inner or outer tube at one point. Thus, it is desirable to fix the muscle member at a position of fixation in the vicinity of the fore distal end of the inner or outer tube. Otherwise, each muscle member may be fixed to the inner or outer tube at a point between its fore end and a middle portion of its length.

Each muscle member should be retained in position fixedly in the circumferential direction of the flexible double tube. For this purpose, it is desirable to provide a restraint member or members on the flexible double tube. For example, a restraint member which is provided with muscle threading grooves is fixedly fitted on the inner or outer tube. In this regard, it is possible to stitch on looped anchor threads for the muscle members. However, it is more desirable to employ restraint rings which are each provided with muscle threading grooves at radial or angular positions on the inner peripheral side. The restraint rings are provided with the muscle threading grooves at a plural number of angularly spaced positions, in a pitch which is necessary for securing stability of the respective muscle members. Further, in a case where the wall thickness of the inner or outer tube, especially, the wall thickness of the inner tube is increased to a certain degree, it becomes possible to provide muscle receptacle grooves axially on the outer peripheral surface of the inner tube to function as restraint grooves, each muscle receptacle groove having a larger width as compared with that of the muscle member. The extent of protrusion of muscle members from the outer peripheral surface of the inner tube can be reduced by accommodating the muscle members in the respective muscle receptacle grooves. However, the muscle receptacle grooves need to be formed shallower as compared with the thickness of the muscle members. The above-described restraint rings can be fitted on to prevent dislodgement of the muscle members from the muscle receptacle grooves. However, the restraint rings can be dispensed with in case the outer tube which surrounds the muscle members is arranged to have a mechanism which functions to prevent dislodgement of the muscle members.

The annular space between the inner and outer tubes is closed at the opposite ends of the flexible double tube to define a hermetically closed space, and a fluid, i.e. a gas or liquid, is introduced into or evacuated from the closed space by a fluid feed means which is connected to the flexible double tube. In this regard, it is desirable for the fluid feed means to use a gas like air.

As described above, the present invention makes it possible to construct a guide tube of a reduced diameter for an endoscope or a surgical tool, which is simple in construction and yet capable of transformation from a flexible structure to a highly rigid structure or vice versa in a secure and reliable manner, by incorporating muscle member into a flexible double tube, which is composed of inner and outer tubes, in such a way as to suppress a diametric difference between the inner and outer tubes.

### BRIEF DESCRIPTION OF THE DRAWINGS

In the accompanying drawings:

FIG. 1 is a schematic outer view of a guide tube fitted on an endoscope;

FIG. 2 is a schematic perspective view of an internal transformable structure of the guide tube;

FIG. 3 is a cross-sectional view taken on line X-X of FIG. 2;

FIG. 4 is a fragmentary sectional view taken on line Y-Y of FIG. 2;

FIG. 5 is a perspective view of a muscle member;

FIG. 6 is a front view of a restraint member;

FIG. 7 is a schematic view explanatory of transformation of the guide tube from a flexible structure to a highly rigid structure;

FIG. 8 is a schematic sectional view of a muscle member in a second embodiment of the invention;

FIG. 9 is a schematic perspective view of a guide tube in a third embodiment of the invention; and

FIG. 10 is a schematic perspective view of a guide tube in a fourth embodiment of the invention.

### DESCRIPTION OF PREFERRED EMBODIMENTS

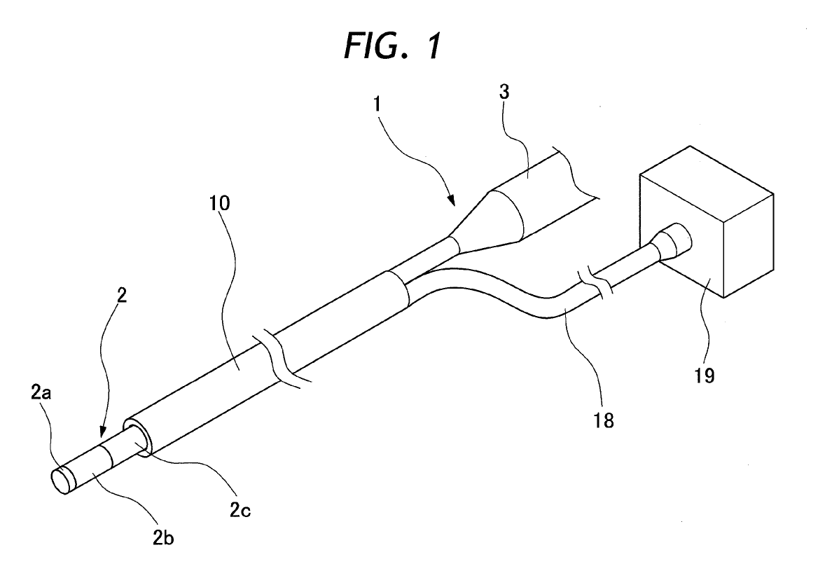

Hereafter, the present invention is described more particularly by way of its preferred embodiments. Needless to say, the present invention should not be construed as being limited to the particular embodiments shown. Referring first to FIG. 1, there is shown an outer view of a guide tube which is fitted on an endoscope. In FIG. 1, indicated at 1 is an endoscope having an elongated insertion rod 2 extended forward from a manipulating head grip 3 for insertion into a body cavity of a patient or examinee. From a fore distal end, the insertion rod 2 is composed of a rigid tip end section 2a, an articularly bendable section 2b, and an elongated flexible body section 2c. The flexible body section 2c of the insertion rod 2 is flexible in bending directions. Provided on the rigid tip end section 2a are an illumination light projection means to illuminate an intracavitary site of interest, and an observation window to capture picture images of the intracavitary site under illumination light. Further, an outlet of a surgical or other medical treating tool is opened at the distal end of the insertion rod 2. The endoscope 1 itself is constructed in a known manner, and therefore detailed descriptions in this regard are omitted here.

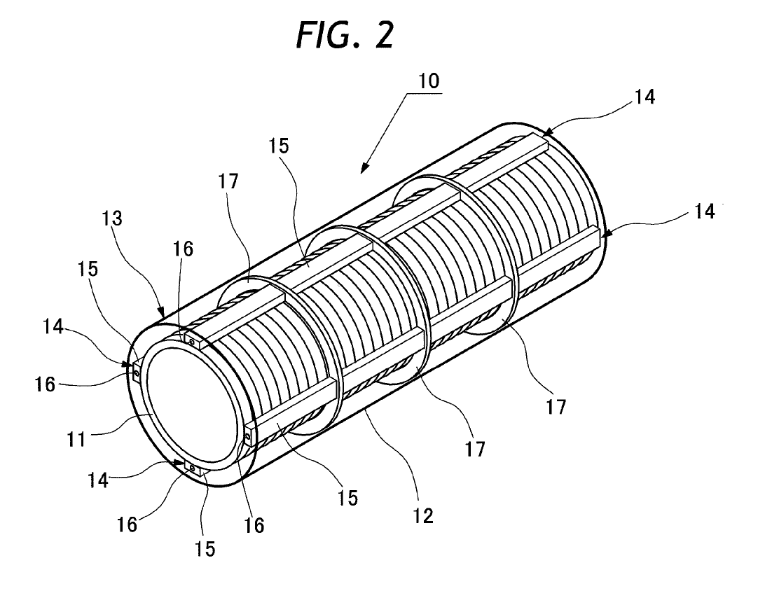

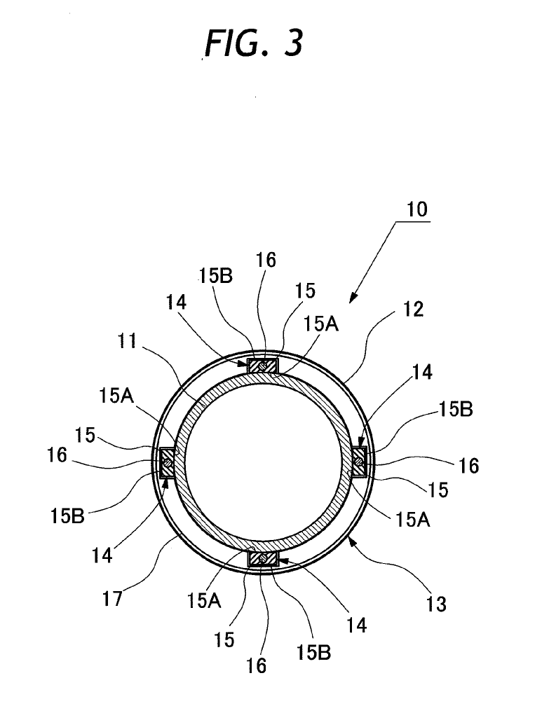

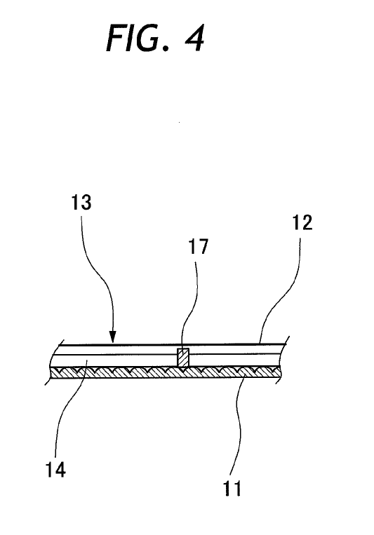

Referring to FIGS. 2 and 3, indicated at 10 is a guide tube in the form of a flexible double tube 13 composed of a flexible inner tube 11 which has open fore distal end and rear or proximal end and which is extended along a longitudinal axis flexibly in bending directions, and an outer tube 12 which is disposed in such a way as to enshroud the inner tube 11 and directly or indirectly attached to the fore and rear ends of the inner tube 11 and extended flexibly in bending directions. Interposed between the inner and outer tubes 11 and 12 are four muscle members 14 at four angularly spaced positions around the outer and inner peripheries of the inner and outer tubes 11 and 12. As shown particularly in FIG. 4, the inner tube 11 is formed of a flexible synthetic resin material and composed of a series of successively connected ring members of a semi-circular shape in cross section. Namely, the inner tube 11 is so arranged as to form a series of cylindrical ridges in the axial direction, alternately providing a grooved first radius portion having a predetermined outside diameter and a ridged second radius portion having a larger outside diameter. The inner tube 11 has a smooth surface on the inner peripheral side. The inner tube 11 which is arranged in this manner has properties such as high flexibility in bending directions and no or low stretchability, in addition to high anti-crushability.

The outer tube 12 is in the form of a non-stretchable thin-wall tube with a slippery surface on the outer side to be directly exposed to a body cavity of a patient, along with high resistance to chemicals. In contrast, the inner surface of the outer tube 12 may be roughened to some extent to insure a securer grip by the muscle members 14 as described hereinlater or, if desired, it may be smoothened out from the standpoint of intimate contact with the muscle members 14.

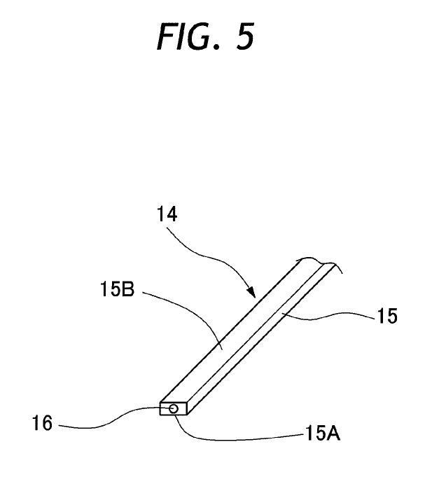

As shown in FIG. 5, the muscle members 14 are each constituted by a friction strip 15 formed of a resilient material like rubber substantially in a rectangular shape in cross section. A metal wire 16 is embedded in the resilient friction strip 15 to serve as a strength retention member. Each friction strip 15 is disposed face to face with the outer periphery of the inner tube 11 on the side of its inner surface 15A and with the inner periphery of the outer tube 12 on the side of its outer surface 15B. In the particular embodiment shown, four muscle members 14 are located at four equidistant angular positions around the inner tube 11. Each muscle member 15 is securely fixed to an outer peripheral surface of the inner tube 11 up to a median point from its fore end or from a point near its fore end, and extended axially toward the proximal end of the flexible double tube 13. Further, each muscle member 14 is formed shorter than the flexible double tube 13. By this difference in length, when the guide tube 10 is bent along a turn in the path of insertion, the muscle member 14 is prevented from protruding beyond the proximal end of the guide tube 10, which would otherwise occur due to a difference in radius of curvature between an angle of curvature on the inner peripheral side and an angle of curvature at the center of the guide tube 10. The extent of this difference in length, necessary for absorption of the protrusive displacement of the muscle member 14, varies depending upon the angles of turns in the path of insertion of the guide tube 10.

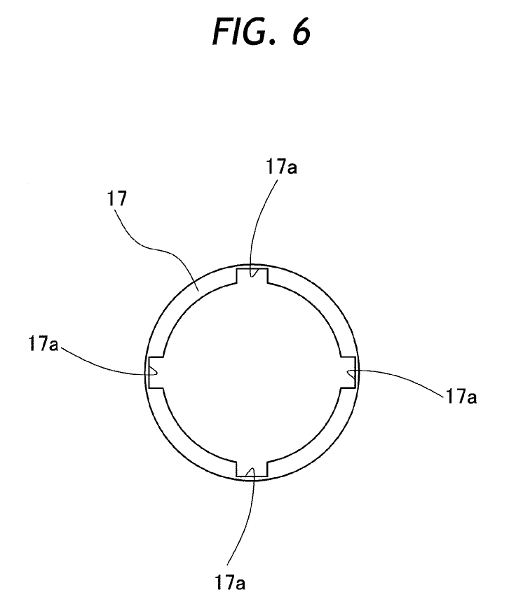

As mentioned hereinbefore, the guide tube 10 is provided with four muscle members 14 which are located with a phase shift of 90 degrees from each other and retained in that position to prevent spontaneous movements in the circumferential direction. To this end, the double tube 13 is provided with a plural number of restraint rings 17 which are located at predetermined intervals in the axial direction and fixed to an outer surface of the inner tube 11. Preferably, the restraint rings 17 are formed of a lightweight and rigid synthetic resin material and are each formed with muscle threading notches 17a at intervals of 90 degrees around the inner periphery for passing the muscle members 14 therethrough, as shown in FIG. 6. Each one of the muscle threading notches 17a is slightly larger in width and in depth as compared with the width and thickness of the muscle member 14. Therefore, the muscle members 14 are restrained of movements in the circumferential direction by the restraint rings 17 but not restrained of movements in the axial direction.

The inner and outer tubes 11 and 12 of the guide tube 10 are securely fixed to each other at the fore and proximal ends in such a way as to form a hermetically closed space therebetween. It is in this closed space that the muscle members 14 and restraint rings 17 are located. As shown in FIG. 1, a suction tube 18 from a pump unit 19 is connected to the closed space.

Being constructed in the manner as described above, the guide tube 10 is introduced into a body cavity along a predetermined path of insertion or by piercing a hole in an intracavitary wall. Anyway, the guide tube 10 is not inserted into a body cavity alone, but it is fitted on and assembled with an endoscope 1 prior to insertion. This is because the endoscope 1 is necessary for confirmation of a path of insertion within a body cavity. The guide tube 10 is fitted on the endoscope 1 in such a way that the rigid tip end section 2a of the insertion rod 2 is located at the fore distal end of the guide tube 10 or in such a way that the insertion rod 2 is projected out of the guide tube 10 up to the articular bending section 2b. By so fitting, a forward view field in the direction of insertion can be taken through the observation window which is provided on the rigid tip end section 2a alongside an illumination window.

The articular bending section 2b of the endoscopic insertion rod 2 can be turned toward an arbitrary direction, and the guide tube 10 is also bendable by way of the muscle members 14 which are interposed between the inner and outer tubes of the flexible double tube 13. Thus, the guide tube 10 as well as the endoscope 1 which is fitted in the guide tube 10 can be smoothly passed along a path of insertion in a body cavity.

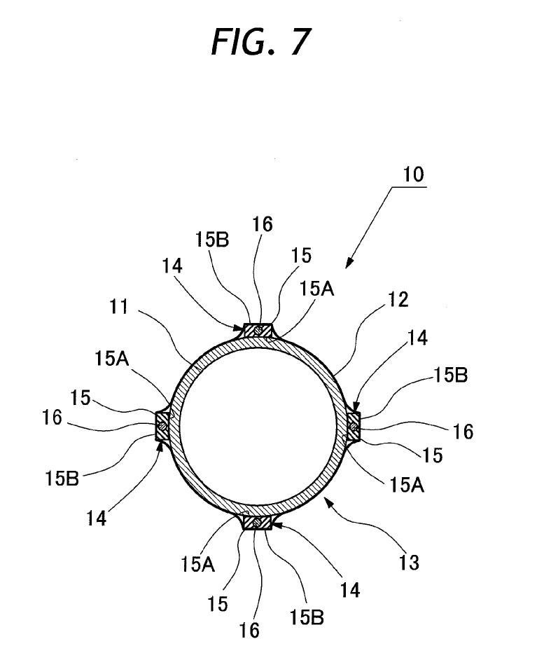

When it becomes necessary to hold the insertion rod 2 in a fixed state at a certain position within the guide tube 10 for the purpose of making an inspection or giving a treatment, the suction pump 19 is activated to evacuate the hermetically closed space between the inner and outer tubes 11 and 12 through the suction tube 18. Whereupon, as shown in FIG. 7, the outer tube 12 of the guide tube 10 is pulled radially inward to fit tightly on the inner tube 11. At this time, the outer tube 12 is pushed against the outer surfaces 15B of the friction strips 15. As a consequence, inner surfaces 15A of the friction strips 15 are stuck fast to the outer periphery of the inner tube 11. That is, the surfaces 15A on the inner side of the friction strips 15 function as coupling surfaces. In this instance, the friction strips 15 are formed of a resilient material like rubber, and the inner tube 11 is formed of a flexible material. Accordingly, the coupling surfaces 15A on the inner side of the friction strips 15 are pressed against an outer peripheral surface of the inner tube 11 to hold the friction strips 15 in a restrained state. As a result, the muscle members 14 are fixed fast to the inner and outer tubes 11 and 12 by frictional engagement therewith.

Since a metal wire 16 is embedded in each one of the muscle members 14 as a strength retention member, flexibility of the inner tube 11 in bending directions is lowered when the muscle members 14 pressed against the inner tube 11. As a consequence, the guide tube 10 as a whole is transformed into a highly rigid structure to ensure stability in bending directions of the endoscopic insertion rod 2 within the guide tube 10.

Therefore, for example, at the time of propelling forceps or a surgical tool like a high frequency tool out of the tool outlet at the fore distal end of the rigid tip end section 2a of the endoscopic insertion rod 2, it becomes possible to hold the rigid tip end section 2a in a stabilized state to make a treatment with accuracy. When it is intended to apply a force to a mucosa by a surgical tool, it has been the usual experience that the endoscope itself is easily moved by a reaction force, making it difficult to apply a force appropriately. The transformation to a highly rigid structure makes it possible for the guide tube 10 to suppress movements of the endoscope at the time of applying a force with an endoscopically inserted tool.

For example, at the time of advancing the insertion rod 2 of the endoscope 1 along a path which is newly opened by stomach perforation, the suction pump 19 is turned off to permit introduction of air into the guide tube 10. Whereupon, the guide tube 10 restores flexibility in bending directions, and can be advanced along the newly opened path of insertion.

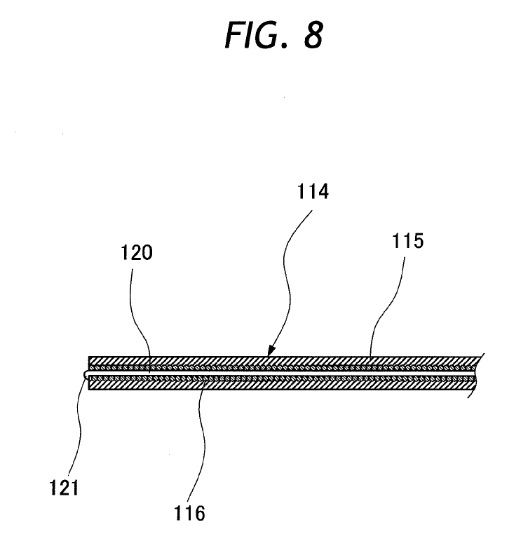

In the above-described embodiment, the guide tube 10 is transformed into a highly rigid structure when it is in a curved shape along a sinuous path of insertion, without permitting adjustments of its curvature. Muscle members 114 of FIG. 8 can be employed for transforming the guide tube 10 into a highly rigid structure after straightening up the guide tube 10 into a substantially rectilinear form. As clear from that figure, the muscle member 114 is basically constituted by a friction strip 115 in the same manner as in the foregoing first embodiment. In this embodiment, however, a tightly wound coil is embedded in each one of the friction strips 115 as a hollow strength retention member 116, instead of the above-described metal wire. A manipulation wire 120 is threaded through the hollow strength retention member 116, with a fore distal end of the manipulation wire 120 fixed to a fore distal end of the hollow strength retention member 116 by adhesion or brazing. On the other hand, the proximal end of the manipulation wire 120 is led out of the proximal end of the hollow strength retention member 116 and then to the outside through a sealed portion between the inner and outer tubes 11 and 12.

By adopting the arrangements just described, the muscle members 114 function in the same manner as the muscle members 14 in the foregoing embodiment when the manipulation wires 120 are put in a free state. In order to straighten out a curved path of insertion at the time of transformation of the guide tube 10 to a highly rigid structure, what is required for an operator is to pull a manipulation wire 120 in a hollow strength retention member 116 which is located on the outer peripheral side of the curvature. By so doing, the guide tube 10 is straightened up thereby to straighten out a sinuous path of insertion into a rectilinear form. The guide tube of this construction is useful particularly when straightening out the sigmoid in lower endoscopy.

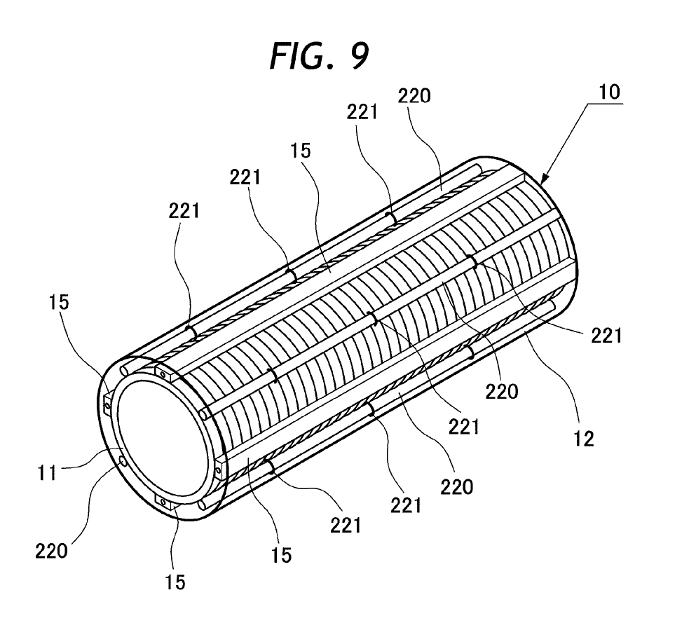

In case metal wires 16 are employed for the strength retention members, as shown in FIG. 9, manipulation wires 220 may be provided at radial positions between the respective muscle members 15. The manipulation wires 220 are retained in the respective radial positions by means looped anchor threads 221 which are provided on the outer periphery of the inner tube 11 at a number of positions along the length of each manipulation wire 220. Thus, the respective manipulation wires 220 can be pushed or pulled in an axial direction within the looped anchor threads 221. In this case, a curved path of insertion can also be straightened out when the guide tube 10 as a whole is transformed into a highly rigid structure. Of course, fore ends of the manipulation wires 220 are securely fixed to the fore distal end or fore distal end portions of the inner tube, while proximal ends of the wires 220 are led out through sealed portions between the inner and outer tubes 11 and 12.

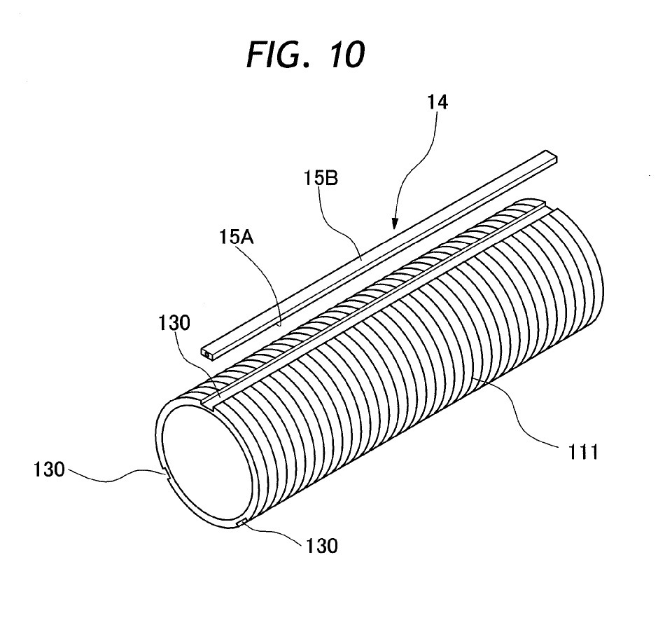

Further, shown in FIG. 10 is another embodiment in which an inner tube 111 is arranged to perform the function of retaining the muscle members 14 (or muscle members 114 of the second embodiment) in the respective radial positions, without using the restraint rings 17 of the first embodiment. As described hereinbefore, the inner tube 111 is provided with an outer peripheral surface having a cylindrical ridge alternately with an annular groove. In this case, three muscle member restraint grooves 120 are formed axially on the outer peripheral surface of the inner tube 111 by cutting outer portions of the cylindrical ridges. Each restraint groove 130 is formed to have a width and a depth which are slightly larger and smaller as compared with the width and thickness of the muscle member 14, respectively.

With the arrangements just described, the muscle members 14 are received in the restraint grooves 130 to prevent spontaneous dislodgements from the respective radial positions. Besides, by accommodation of the muscle members 14 in the restraint grooves 130, it becomes possible to reduce the outside diameter of the guide tube as a whole. In this case, however, if the muscle members 14 are sunken in the restraint grooves 130, it becomes difficult for the outer tube to press the surfaces 14B of the muscle members 14 at the time of evacuation of the hermetically sealed space. Therefore, the restraint grooves 130 should be formed shallower as compared with the thickness of the muscle members 14, letting the latter project radially outward beyond the ridges of the inner tube 111.

In this instance, the muscle members 14 may be simply placed in the restraint grooves 130 is their dislodgements from the respective radial positions are securely restrained by the grooves 130. If not, it is desirable to provide the restraint ring of FIG. 6 at a number of axially spaced positions to restrain dislodgements of the muscle members 14 in an assured manner.

What is claimed is:

1. A guide tube for guiding an endoscope or a surgical tool in or into a body cavity, comprising:
an inner tube having open fore and rear ends and a longitudinal axis, along with flexibility in bending directions;
an outer tube disposed to enshroud said inner tube and directly or indirectly connected to said fore and rear ends of said inner tube, said outer tube having flexibility in bending directions;
at least one elongated muscle member located in a hermetically closed space defined between said inner and outer tubes in parallel relation with said longitudinal axis of said inner tube, and having an engaging surface on inner or outer side to be brought into pressed engagement with at least one of said inner and outer tubes, said muscle member being attached to an elongated strength retention member having no elasticity in a longitudinal direction but having flexibility in bending directions;
a restraint member provided at least on one of said inner and outer tubes and adapted to restrain said muscle member of dislodgement in a circumferential direction about said longitudinal axis from a predetermined angular or radial position on said inner tube; and
a fluid evacuation means connected to said guide tube for communication with said hermetically closed space between said inner and outer tubes, said evacuation means being switched on to evacuate a fluid from said closed space, bringing said engaging surface into pressed engagement with said inner or outer tube to transform said guide tube into a highly rigid structure, and switched off at the time of restoring flexibility of said guide tube by introduction of a fluid in said closed space.

2. A guide tube as set forth in claim 1, wherein said inner tube having an outer peripheral surface alternately formed with a first radius portion in the form of an annular groove having a predetermined outside diameter and a second radius portion in the form of a cylindrical ridge having a larger outside diameter than said first radius portion.

3. A guide tube as set forth in claim 1, wherein said outer tube is constituted by a non-elastic tube.

4. A guide tube as set forth in claim 1, wherein said muscle member is located at four radial or angular positions around said longitudinal axis of said inner tube with a phase shift of 90 from each other.

5. A guide tube as set forth in claim 1, wherein said engaging surface is of a resilient material.

6. A guide tube as set forth in claim 1, wherein said strength retention member is constituted by a metal wire of a round shape in cross section, embedded in said muscle member.

7. A guide tube as set forth in claim 1, wherein said muscle member is shorter in length as compared with said inner and outer tubes, and disposed in a free state at least at one end thereof.

8. A guide tube as set forth in claim 1, further comprising a manipulation wire in association with said inner or outer tube, said manipulation wire having a fore distal end thereof securely fixed to a fore distal end of said inner or outer tube and having the other end extended as far as a proximal base end of said inner tube.

9. A guide tube as set forth in claim 1, wherein said restraint member is in the form of an annular ring having notched grooves at predetermined angular positions on inner periphery thereof for threading muscle members therethrough, said restraint member being provided at a plural number of positions at predetermined intervals in axial direction of said inner tube.

10. A guide tube as set forth in claim 1, wherein said restraint member is constituted by a restraint groove formed axially on outer periphery of said inner tube, said restraint groove having a larger width and a shallower depth as compared with width and thickness of said muscle member, respectively.

11. A guide tube as set forth in claim 1, wherein said fluid evacuation means is adapted to evacuate a gas from said closed space.
